(19) United States
(12) Patent Application Publication
(10) Pub. No.: US 2015/0141726 A1
(43) Pub. Date: May 21, 2015

(54) PROCESS FOR PRODUCING OLEFINS FROM A COAL FEED

(71) Applicant: UOP LLC, Des Plaines, IL (US)

(72) Inventors: Vasant P. Thakkar, Elk Grove Village, IL (US); Paul T. Barger, Arlington Heights, IL (US); Maureen L. Bricker, Buffalo Grove, IL (US); John Q. Chen, Glenview, IL (US); Peter K. Coughlin, Mundelein, IL (US); Stanley J. Frey, Palatine, IL (US); James A. Johnson, Burr Ridge, IL (US); Joseph A. Kocal, Glenview, IL (US); Matthew Lippmann, Chicago, IL (US); Kurt M. Vanden Bussche, Lake in the Hills, IL (US)

(21) Appl. No.: 14/467,764

(22) Filed: Aug. 25, 2014

Related U.S. Application Data
(60) Provisional application No. 61/906,073, filed on Nov. 19, 2013.

(51) Int. Cl. C07C 4/06; C07C 7/10; C07C 7/12; C07C 7/09; C07C 7/13; C07C 7/04; C07C 2/06

(52) U.S. Cl. CPC C07C 4/06; C07C 7/04; C07C 7/10; C07C 2/06; C07C 7/09; C07C 7/13; C07C 7/12; C07C 2529/89; C07C 2523/28; C07C 2523/755; C07C 2523/30
USPC 585/330; 585/324

(57) ABSTRACT

A process for producing olefins from a coal feed includes providing a coal tar stream and fractionating the coal tar stream to provide a hydrocarbon stream that includes hydrocarbons having an initial boiling point of about 250° C. or greater. The hydrocarbon stream is hydrotreated to reduce a concentration of one or more of nitrogen, sulfur, and oxygen in the hydrocarbon stream, and the hydrotreated hydrocarbon stream is cracked in a fluidized catalytic cracking zone to produce an olefin stream.

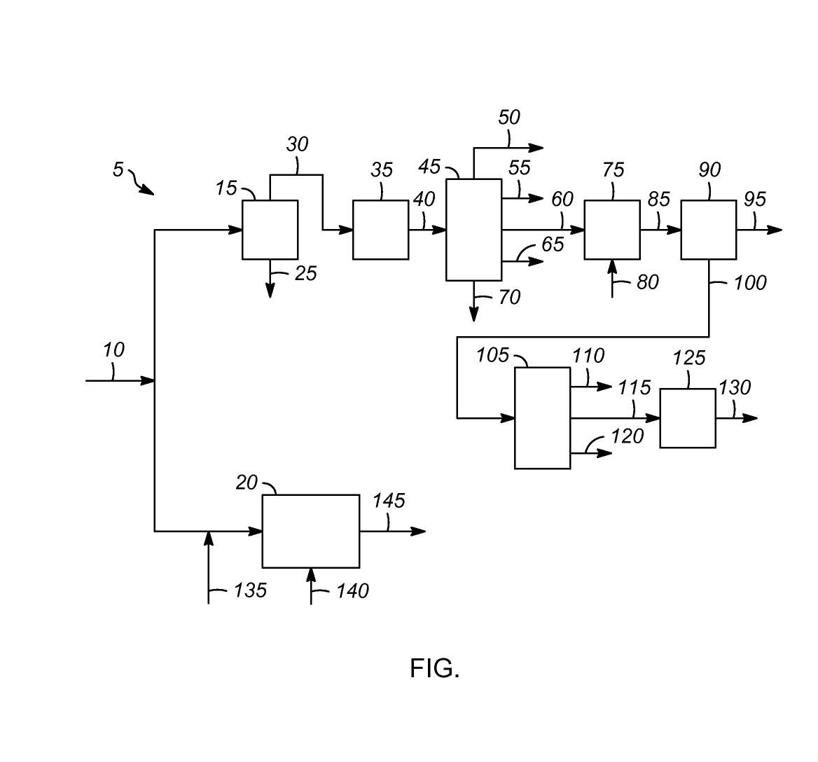

### RELATED APPLICATIONS

This application claims priority to U.S. Provisional Application No. 61/906,073 filed on Nov. 19, 2013, the entirety of which is incorporated herein by reference.

### BACKGROUND OF THE INVENTION

Many different types of chemicals are produced from the processing of petroleum. However, petroleum is becoming more expensive because of increased demand in recent decades.

Therefore, attempts have been made to provide alternative sources for the starting materials for manufacturing chemicals. Attention is now being focused on producing liquid hydrocarbons from solid carbonaceous materials, such as coal, which is available in large quantities in countries such as the United States and China.

Pyrolysis of coal produces coke and coal tar. The coke-making or “coking” process consists of heating the material in closed vessels in the absence of oxygen to very high temperatures. Coke is a porous but hard residue that is mostly carbon and inorganic ash, which may be used in making steel.

Coal tar is the volatile material that is driven off during heating, and it comprises a mixture of a number of hydrocarbon compounds. It can be separated to yield a variety of organic compounds, such as benzene, toluene, xylene, naphthalene, anthracene, and phenanthrene. These organic compounds can be used to make numerous products, for example, dyes, drugs, explosives, flavorings, perfumes, preservatives, synthetic resins, and paints and stains. The residual pitch left from the separation is used for paving, roofing, waterproofing, and insulation.

Olefins are desirable products in the petrochemical industry. Thus, there is a need for a process for producing olefins from a coal feed.

### SUMMARY OF THE INVENTION

In one aspect, a process for producing olefins from a coal feed includes providing a coal tar stream and fractionating the coal tar stream to provide a hydrocarbon stream that includes hydrocarbons having an initial boiling point of about 250° C. or greater. The hydrocarbon stream is hydrotreated to reduce a concentration of one or more of nitrogen, sulfur, and oxygen in the hydrocarbon stream, and the hydrotreated hydrocarbon stream is cracked in a fluidized catalytic cracking zone to produce an olefin stream.

In another aspect, a process for producing olefins from a coal feed includes pyrolyzing coal to produce a coke stream and a coal tar stream. The process further includes separating the coal tar stream to produce a hydrocarbon stream excluding a pitch fraction and hydrotreating the hydrocarbon stream to produce a hydrotreated hydrocarbon stream having reduced concentration of one or more of nitrogen, sulfur, and oxygen. The hydrotreated hydrocarbon stream is cracked in a fluidized catalytic cracking zone to produce an olefin stream.

In a third aspect, a process for producing olefins from a coal feed, includes pyrolyzing coal to produce a coke stream and a coal tar stream and separating the coal tar stream to produce a hydrocarbon stream excluding a pitch fraction. The hydrocarbon stream is hydrotreated to produce a hydrotreated hydrocarbon stream having reduced concentration of one or more of nitrogen, sulfur, and oxygen. The process further includes cracking the hydrotreated hydrocarbon stream in a fluidized catalytic cracking zone to produce an olefin stream comprising a plurality of olefins, and separating the olefin stream into a plurality of olefin product streams based on a carbon number of the olefins.

### BRIEF DESCRIPTION OF THE DRAWING

The FIGURE illustrates one embodiment of the process of the present invention.

### DETAILED DESCRIPTION OF THE INVENTION

The FIGURE shows one embodiment of an olefin producing process 5. A coal feed 10 can be sent to either a pyrolysis zone 15 or a gasification zone 20. Alternatively, the coal feed 10 can be split into two parts and sent to both.

In the pyrolysis zone 15, the coal feed 10 is heated at high temperature, e.g., up to about 2,000° C. (3,600° F.), in the absence of oxygen to drive off the volatile components. Coking produces a coke stream 25 and coal tar stream 30. The coke from the coke stream 25 can be used in other processes, such as the manufacture of steel.

The coal tar stream 30 which comprises the volatile components from the coking process can be sent to an optional contamination removal zone 35, if desired.

The contaminant removal zone 35 for removing one or more contaminants from the coal tar stream or another process stream may be located at various positions along the process depending on the impact of the particular contaminant on the product or process and the reason for the contaminant's removal, as described further below. For example, the contaminant removal zone 35 can be positioned upstream of a separation zone 45. Some contaminants have been identified to interfere with a downstream processing step or hydrocarbon conversion process, in which case the contaminant removal zone 35 may be positioned upstream of the separation zone 45 or between the separation zone 45 and the particular downstream processing step at issue. Still other contaminants have been identified that should be removed to meet particular product specifications. Where it is desired to remove multiple contaminants from the hydrocarbon or process stream, various contaminant removal zones 35 may be positioned at different locations along the process. In still other approaches, a contaminant removal zone 35 may overlap or be integrated with another process within the system, in which case the contaminant may be removed during another portion of the process, including, but not limited to the separation zone 45 or the downstream hydrocarbon conversion zone. This may be accomplished with or without modification to these particular zones, reactors or processes. While the contaminant removal zone 35 is often positioned downstream of the hydrocarbon conversion reactor, it should be understood that the contaminant removal zone 35 in accordance herewith may be positioned upstream of the separation zone 45, between the separation zone 45 and the hydrocarbon conversion zone, or downstream of the hydrocarbon conversion zone or along other streams within the process stream, such as, for example, a carrier fluid stream, a fuel stream, an oxygen source stream, or any streams used in the systems and the processes described herein. The contaminant concentration is controlled by removing at least a portion of the contaminant from the coal tar stream 30. As used herein, the term removing may refer to actual removal, for example by adsorption, absorption, or membrane separation, or it may refer to conversion of the contaminant to a more tolerable compound, or both.

The decontaminated coal tar feed 40 is sent to a separation zone 45 where it is separated into two or more fractions50, 55, 60, 65, 70. Coal tar comprises a complex mixture of heterocyclic aromatic compounds and their derivatives with a wide range of boiling points. The number of fractions and the components in the various fractions can be varied as is well known in the art. A typical separation process involves separating the coal tar into four to six streams. For example, there can be a fraction comprising NH₃, CO, and light hydrocarbons, a light oil fraction with boiling points between 0° C. and 180° C., a middle oil fraction with boiling points between 180° C. to 230° C., a heavy oil fraction with boiling points between 230 to 270° C., an anthracene oil fraction with boiling points between 270° C. to 350° C., and pitch.

The light oil fraction contains compounds such as benzenes, toluenes, xylenes, naphtha, coumarone-indene, dicyclopentadiene, pyridine, and picolines. The middle oil fraction contains compounds such as phenols, cresols and cresylic acids, xylenols, naphthalene, high boiling tar acids, and high boiling tar bases. The heavy oil fraction contains benzene absorbing oil and creosotes. The anthracene oil fraction contains anthracene. Pitch is the residue of the coal tar distillation containing primarily aromatic hydrocarbons and heterocyclic compounds.

As illustrated, the coal tar feed 40 is separated into a gas fraction 50 containing gases such as NH₃ and CO as well as light hydrocarbons, such as ethane, hydrocarbon fractions 55, 60, and 65 having different boiling point ranges, and a pitch fraction 70.

Suitable separation processes include, but are not limited to fractionation, such as a distillation, solvent extraction, and adsorption.

One or more of the fractions 50, 55, 60, 65, 70 can be further processed, as desired. As illustrated in the FIGURE, fraction 60 can be sent to a hydrotreating zone 75. The fraction 60 preferably includes relatively heavy hydrocarbons having initial boiling points greater than 250° C., preferably in the range of about 300° C. to about 400° C., although other suitable fractions may be selected as is known in the art. Hydrotreating is a process in which hydrogen gas 80 is contacted with a hydrocarbon stream in the presence of suitable catalysts which are primarily active for the removal of heteroatoms, such as sulfur, nitrogen, oxygen, and metals from the hydrocarbon feedstock. In this context, removal includes actual removal of at least a portion of the heteroatoms. For example, the hydrotreating process preferably reduces a concentration of sulfur to 50 parts per million or less. Similarly, the hydrotreating preferably reduces a concentration of nitrogen to about 20 parts per million or less. In hydrotreating, hydrocarbons with double and triple bonds may be saturated. Aromatics may also be saturated. Typical hydrotreating reaction conditions include a temperature of about 290° C. (550° F.) to about 455° C. (850° F.), a pressure of about 3.4 MPa (500 psig) to about 27.6 MPa (4,000 psig), a liquid hourly space velocity of about 0.1 .hr⁻¹ to about 5 hr⁻¹, and a hydrogen rate of about 168 to about 1,685 Nm³/m³ oil (1,000 to 10,000 scf/bbl). Typical hydrotreating catalysts include at least one Group VIII metal, preferably iron, cobalt and nickel, and at least one Group VI metal, preferably molybdenum and tungsten, on a high surface area support material, preferably alumina. Other typical hydrotreating catalysts include zeolitic catalysts, as well as noble metal catalysts where the noble metal is selected from palladium and platinum. The hydrotreated hydrocarbon stream 85 is then routed to a cracking zone 90.

Cracking is a process in which a bond between carbon atoms in a hydrocarbon is broken or “cracked” to form lower molecular weight hydrocarbon compounds. The cracking zone 90 can take multiple forms, including, for example, a hydrocracking zone or a fluid catalytic cracking zone.

Hydrocracking is a process in which hydrocarbons crack in the presence of hydrogen to lower molecular weight hydrocarbons. Typical hydrocracking conditions may include a temperature of about 290° C. (550° F.) to about 468° C. (875° F.), a pressure of about 3.5 MPa (500 psig) to about 20.7 MPa (3,000 psig), a liquid hourly space velocity (LHSV) of about 0.5 to less than about 5 hr⁻¹, and a hydrogen rate of about 421 to about 2,527 Nm³/m³ oil (2,500 to 15,000 scf/bbl). Typical hydrocracking catalysts include amorphous silica-alumina bases or low-level zeolite bases combined with one or more Group VIII or Group VIB metal hydrogenating components, or a crystalline zeolite cracking base upon which is deposited a Group VIII metal hydrogenating component. Additional hydrogenating components may be selected from Group VIB for incorporation with the zeolite base.

Fluid catalytic cracking (FCC) is a catalytic hydrocarbon conversion process accomplished by contacting heavier hydrocarbons in a fluidized reaction zone with a catalytic particulate material. The reaction in catalytic cracking is carried out in the absence of substantial added hydrogen or the consumption of hydrogen. The process typically employs a powdered catalyst having the particles suspended in a rising flow of feed hydrocarbons to form a fluidized bed. In representative processes, cracking takes place in a riser, which is a vertical or upward sloped pipe. Typically, a pre-heated feed is sprayed into the base of the riser via feed nozzles where it contacts hot fluidized catalyst and is vaporized on contact with the catalyst, and the cracking occurs converting the high molecular weight oil into lighter components including liquefied petroleum gas (LPG), gasoline, and a distillate. The catalyst-feed mixture flows upward through the riser for a short period (a few seconds), and then the mixture is separated in cyclones. The hydrocarbons are directed to a fractionator for separation into LPG, gasoline, diesel, kerosene, jet fuel, and other possible fractions. While going through the riser, the cracking catalyst is deactivated because the process is accompanied by formation of coke which deposits on the catalyst particles. Contaminated catalyst is separated from the cracked hydrocarbon vapors and is further treated with steam to remove hydrocarbon remaining in the pores of the catalyst. The catalyst is then directed into a regenerator where the coke is burned off the surface of the catalyst particles, thus restoring the catalyst's activity and providing the necessary heat for the next reaction cycle. The process of cracking is endothermic. The regenerated catalyst is then used in the new cycle. Typical FCC conditions include a temperature of about 400° C. to about 800° C., a pressure of about 0 to about 688 kPag (about 0 to 100 psig), and contact times of about 0.1 seconds to about 1 hour. The conditions are determined based on the hydrocarbon feedstock being cracked, and the cracked products desired. Zeolite-based catalysts are commonly used in FCC reactors, as are composite catalysts which contain zeolites, silica-aluminas, aluminas, and other binders. For example, the cracking catalyst may include one or more of an active amorphous clay type catalyst, a high activity crystalline molecular sieve, and an MFI zeolite.

The cracking produces an olefin stream 100 which may be routed from the cracking zone 90 to an olefin separation zone 105 to separate the olefin stream 100 into two or more product streams. The olefin separation zone 105 can include one or more of a distillation zone, a solvent extraction zone, or a fractionation zone. The olefin stream 100 is separated into multiple product streams 110, 115, 120 based on the number of carbon atoms in the olefins. As shown in the FIGURE, the stream 105 is separated into three product streams 110, 115, 120, although it will be appreciated that the stream 105 can be divided into more or fewer streams without departing from the scope of the invention. The olefin product streams 110, 115, 120 may be collected as end products, or may be subject to additional downstream processing, as is known in the art. As an example, the FIGURE shows a product stream 115 that includes C₂ olefins is subject to additional processing. The stream 115 is routed to a polymerization zone 125. The stream 115 is polymerized to produce higher molecular weight hydrocarbons having a higher carbon number.

In addition, the hydrocarbon streams 55, 65 of the separation zone 45 and the olefin streams 110, 120 of the olefin separation zone 105 may be also subject to additional processing including, but not limited to, transalkylation, alkylation, oxidation, hydrogenation processing.

Transalkylation is a chemical reaction resulting in transfer of an alkyl group from one organic compound to another. Catalysts, particularly zeolite catalysts, are often used to effect the reaction. If desired, the transalkylation catalyst may be metal stabilized using a noble metal or base metal, and may contain suitable binder or matrix material such as inorganic oxides and other suitable materials. In a transalkylation process, a polyalkylaromatic hydrocarbon feed and an aromatic hydrocarbon feed are provided to a transalkylation reaction zone. The feed is usually heated to reaction temperature and then passed through a reaction zone, which may comprise one or more individual reactors. Passage of the combined feed through the reaction zone produces an effluent stream comprising unconverted feed and product monoalkylated hydrocarbons. This effluent is normally cooled and passed to a stripping column in which substantially all C₅ and lighter hydrocarbons present in the effluent are concentrated into an overhead stream and removed from the process. An aromatics-rich stream is recovered as net stripper bottoms, which is referred to as the transalkylation effluent.

The transalkylation reaction can be effected in contact with a catalytic composite in any conventional or otherwise convenient manner and may comprise a batch or continuous type of operation, with a continuous operation being preferred. The transalkylation catalyst is usefully disposed as a fixed bed in a reaction zone of a vertical tubular reactor, with the alkylaromatic feed stock charged through the bed in an upflow or downflow manner. The transalkylation zone normally operates at conditions including a temperature in the range of about 130° C. to about 540° C. The transalkylation zone is typically operated at moderately elevated pressures broadly ranging from about 100 kPa to about 10 MPa absolute. The transalkylation reaction can be effected over a wide range of space velocities. That is, volume of charge per volume of catalyst per hour; weight hourly space velocity (WHSV) generally is in the range of from about 0.1 to about 30 hr⁻¹. The catalyst is typically selected to have relatively high stability at a high activity level.

Alkylation is typically used to combine light olefins, for example mixtures of alkenes such as propylene and butylene, with isobutane to produce a relatively high-octane branched-chain paraffinic hydrocarbon fuel, including isoheptane and isooctane. Similarly, an alkylation reaction can be performed using an aromatic compound such as benzene in place of the isobutane. When using benzene, the product resulting from the alkylation reaction is an alkylbenzene (e.g. toluene, xylenes, ethylbenzene, etc.). For isobutane alkylation, typically, the reactants are mixed in the presence of a strong acid catalyst, such as sulfuric acid or hydrofluoric acid. The alkylation reaction is carried out at mild temperatures, and is typically a two-phase reaction. Because the reaction is exothermic, cooling is needed. Depending on the catalyst used, normal refinery cooling water provides sufficient cooling. Alternatively, a chilled cooling medium can be provided to cool the reaction. The catalyst protonates the alkenes to produce reactive carbocations which alkylate the isobutane reactant, thus forming branched chain paraffins from isobutane. Aromatic alkylation is generally now conducted with solid acid catalysts including zeolites or amorphous silica-aluminas.

The alkylation reaction zone is maintained at a pressure sufficient to maintain the reactants in liquid phase. For a hydrofluoric acid catalyst, a general range of operating pressures is from about 200 to about 7,100 kPa absolute. The temperature range covered by this set of conditions is from about −20° C. to about 200° C. For at least alkylation of aromatic compounds, the temperature range is from about 100° C. to about 200° C. at the pressure range of about 200 to about 7,100 kPa.

Oxidation involves the oxidation of hydrocarbons to oxygen-containing compounds, such as aldehydes. The hydrocarbons include alkanes, alkenes, typically with carbon numbers from 2 to 15, and alkyl aromatics, linear, branched, and cyclic alkanes and alkenes can be used. Oxygenates that are not fully oxidized to ketones or carboxylic acids can also be subjected to oxidation processes, as well as sulfur compounds that contain —S—H moieties, thiophene rings, and sulfone groups. The process is carried out by placing an oxidation catalyst in a reaction zone and contacting the feed stream which contains the desired hydrocarbons with the catalyst in the presence of oxygen. The type of reactor which can be used is any type well known in the art such as fixed-bed, moving-bed, multi-tube, CSTR, fluidized bed, etc. The feed stream can be flowed over the catalyst bed either up-flow or down-flow in the liquid, vapor, or mixed phase. In the case of a fluidized-bed, the feed stream can be flowed co-current or counter-current. In a CSTR the feed stream can be continuously added or added batch-wise. The feed stream contains the desired oxidizable species along with oxygen. Oxygen can be introduced either as pure oxygen or as air, or as liquid phase oxidants including hydrogen peroxide, organic peroxides, or peroxy-acids. The molar ratio of oxygen (O₂) to alkane can range from about 5:1 to about 1:10. In addition to oxygen and alkane or alkene, the feed stream can also contain a diluent gas selected form nitrogen, neon, argon, helium, carbon dioxide, steam or mixtures thereof. As stated, the oxygen can be added as air which could also provide a diluent. The molar ratio of diluent gas to oxygen ranges from greater than zero to about 10:1. The catalyst and feed stream are reacted at oxidation conditions which include a temperature of about 300° C. to about 600° C., a pressure of about 101 kPa to about 5,066 kPa and a space velocity of about 100 to about 100,000 hr⁻¹.

Hydrogenation involves the addition of hydrogen to hydrogenatable hydrocarbon compounds. Alternatively hydrogen can be provided in a hydrogen-containing compound with ready available hydrogen, such as tetralin, alcohols, hydrogenated naphthalenes, and others via a transfer hydrogenation process with or without a catalyst. The hydrogenatable hydrocarbon compounds are introduced into a hydrogenation zone and contacted with a hydrogen-rich gaseous phase and a hydrogenation catalyst in order to hydrogenate at least a portion of the hydrogenatable hydrocarbon compounds. The catalytic hydrogenation zone may contain a fixed, ebulated or fluidized catalyst bed. This reaction zone is typically at a pressure from about 689 kPag (100 psig) to about 13,790 kPa gauge (2,000 psig) with a maximum catalyst bed temperature in the range of about 177° C. (350° F.) to about 454° C. (850° F.). The liquid hourly space velocity is typically in the range from about 0.2 hr⁻¹ to about 10 hr⁻¹ and hydrogen circulation rates from about 200 standard cubic feet per barrel (SCFB) (35.6 m³/m³) to about 10,000 SCFB (1778 m³/m³).

In some processes, all or a portion of the coal feed 10 is mixed with oxygen 135 and steam 140 and reacted under heat and pressure in the gasification zone 20 to form syngas 145, which is a mixture of carbon monoxide and hydrogen. The syngas 145 can be further processed using the Fischer-Tropsch reaction to produce gasoline or using the water-gas shift reaction to produce more hydrogen.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention. It being understood that various changes may be made in the function and arrangement of elements described in an exemplary embodiment without departing from the scope of the invention as set forth in the appended claims.

What is claimed is:

1. A process for producing olefins from a coal feed, comprising:
providing a coal tar stream;
fractionating the coal tar stream to provide a hydrocarbon stream comprising hydrocarbons having an initial boiling point of about 250° C. or greater;
hydrotreating the hydrocarbon stream to reduce a concentration of one or more of nitrogen, sulfur, and oxygen in the hydrocarbon stream; and
cracking the hydrotreated hydrocarbon stream in a fluidized catalytic cracking zone to produce an olefin stream.

2. The process of claim 1, wherein hydrotreating the hydrocarbon stream comprises contacting the hydrocarbon stream with a hydrotreating catalyst including a metal function comprising one or more of nickel, molybdenum, cobalt, and tungsten.

3. The process of claim 1, wherein the hydrocarbon stream comprises hydrocarbons having the initial boiling point between about 300° C. and about 400° C.

4. The process of claim 1, wherein the olefin stream comprises a plurality of olefins, the process further comprising:
separating the olefin stream into a plurality of olefin product streams based on a carbon number of the olefins.

5. The process of claim 4, wherein separating the olefin stream comprises distilling the olefin stream.

6. The process of claim 4, wherein separating the olefin stream comprises performing a solvent extraction on the olefin stream.

7. The process of claim 4, wherein separating the olefin stream produces at least a first olefin product stream including C₂ olefins, and further comprising polymerizing said first olefin product stream to produce hydrocarbons of a higher carbon number.

8. The process of claim 1, wherein cracking the hydrotreated hydrocarbon stream comprises contacting the hydrotreated hydrocarbon stream with a cracking catalyst, the cracking catalyst comprising one or more of an active amorphous clay type catalyst, a high activity crystalline molecular sieve, and an MFI zeolite.

9. The process of claim 1, wherein hydrotreating the hydrocarbon stream reduces the concentration of sulfur to less than 50 parts per million, or reduces the concentration of nitrogen to less than 20 parts per million, or both.

10. A process for producing olefins from a coal feed, comprising:
pyrolyzing coal to produce a coke stream and a coal tar stream;
separating the coal tar stream to produce a hydrocarbon stream comprising hydrocarbons having an initial boiling point in a range of about 250° C. to about 400° C.;
hydrotreating the hydrocarbon stream to produce a hydrotreated hydrocarbon stream having reduced concentration of one or more of nitrogen, sulfur, and oxygen; and
cracking the hydrotreated hydrocarbon stream in a fluidized catalytic cracking zone to produce an olefin stream.

11. The process of claim 10, wherein hydrotreating the hydrocarbon stream comprises contacting the hydrocarbon stream with a hydrotreating catalyst including a metal function comprising one or more of nickel, molybdenum, cobalt, and tungsten.

12. The process of claim 10, wherein the hydrocarbon stream comprises hydrocarbons having an initial boiling point between about 300° C. and about 400° C.

13. The process of claim 10, wherein the olefin stream comprises a plurality of olefins, the process further comprising:
separating the olefin stream into a plurality of olefin product streams based on a carbon number of the olefins.

14. The process of claim 13, wherein separating the olefin stream comprises distilling the olefin stream.

15. The process of claim 13, wherein separating the olefin stream comprises performing a solvent extraction on the olefin stream.

16. The process of claim 13, wherein separating the olefin stream produces at least a first olefin product stream comprising C₁ and C₂ hydrocarbons, and further comprising polymerizing said first olefin product stream to produce hydrocarbons of a higher carbon number.

17. The process of claim 10, wherein cracking the hydrotreated hydrocarbon stream comprises contacting the hydrotreated hydrocarbon stream with a cracking catalyst, the cracking catalyst comprising one or more of an active amorphous clay type catalyst, a high activity crystalline molecular sieve, and an MFI zeolite.

18. The process of claim 10, wherein hydrotreating the hydrocarbon stream reduces the concentration of sulfur to less than 50 parts per million, or reduces the concentration of nitrogen to less than 20 parts per million, or both.

19. A process for producing olefins from a coal feed, comprising:
pyrolyzing coal to produce a coke stream and a coal tar stream;
separating the coal tar stream to produce a hydrocarbon stream comprising hydrocarbons having an initial boiling point in a range of about 250° C. to about 400° C.;
hydrotreating the hydrocarbon stream to produce a hydrotreated hydrocarbon stream having reduced concentration of one or more of nitrogen, sulfur, and oxygen; and
cracking the hydrotreated hydrocarbon stream in a fluidized catalytic cracking zone to produce an olefin stream comprising a plurality of olefins;
separating the olefin stream into a plurality of olefin product streams based on a carbon number of the olefins.

20. The process of claim 19, wherein separating the olefin stream produces at least a first olefin product stream comprising C₁ and C₂ hydrocarbons, and further comprising polymerizing said first olefin product stream to produce hydrocarbons of a higher carbon number.
